(19) United States
(12) Patent Application Publication
(10) Pub. No.: US 2012/0219980 A1
(43) Pub. Date: Aug. 30, 2012

(54) SYSTEM AND METHOD FOR PIPETTING GUIDANCE

(75) Inventor: Vijay Pradeep, San Francisco, CA (US)

(73) Assignee: WILLOW GARAGE, INC., Menlo Park, CA (US)

(21) Appl. No.: 13/035,691

(22) Filed: Feb. 25, 2011

(51) Int. Cl. C12Q 1/02; C12M 1/34; G01N 1/00; G01N 33/00

(52) U.S. Cl. 435/29; 422/63; 435/287.1; 436/174

(57) ABSTRACT

Embodiments are described wherein systems and methods for assisting with pipette-based substance transport between two or more trays is disclosed. A controller may be operatively coupled to each of a first matrix of lights and a second matrix of lights and configured to selectively and discretely illuminate one or more of a first matrix of wells and a second matrix of wells subject to a predetermined instruction set contained on a memory device operatively coupled to the controller. The predetermined instruction set may be configured to direct a pipette operator, by discretely illuminating groupings of wells in the first and second matrices of wells, to sample substances from various wells of the first matrix of wells and dispose of them in various wells of the second matrix of wells.

### FIELD OF THE INVENTION

The present invention relates to systems and methods for the precision movement of substances from one tray to another, such as in cell culture and biotechnology laboratory environments.

### BACKGROUND

Many organizations around the world spend significant resource on laboratory testing that involves the movement or transport of substance or specimen portions from a movable reservoir to a particular well in a sample tray, or from a particular well in a source tray to a particular well in a destination tray. Referring to FIGS. 1A and 1B, this practice, also known as “pipetting”, often involves the use of a pipette instrument (2) that has a narrowed distal tip (4) configured to capture and/or eject various substances into and out of particular wells (8) that are formed in the tray (4). Pipetting can be very tedious, and a mistake can be worth millions of dollars to an organization engaged in important research that requires precision pipetting without errors as to source and destination of various substances relative to the pertinent cell trays. FIG. 2 depicts an orthogonal view of a typical sample tray (6) comprising twelve rows of wells (8). The various wells (8) may be located using a Cartesian coordinate system (10), wherein an X axis (12) is associated with one of rows 1-12, as depicted in FIG. 2, and wherein a Y axis (14) is associated with one of the orthogonal rows 1-20. There is a need to assist personnel engaged in repetitive pipetting exercises with their level of precision when transporting substances from source wells to destination wells. Indeed, other applications, such as repetitive disbursal of substances from a pipette reservoir to a designated pattern of wells, also may be assisted with technology suited to assist with the tracking of pipette/well interaction as directed by laboratory personnel.

### BRIEF DESCRIPTION OF THE DRAWINGS

FIGS. 1A and 1B illustrate orthogonal views of conventional pipette and tray configurations.

FIG. 2 illustrates an orthogonal view of a conventional sample tray comprising a plurality of wells organized in rows.

FIGS. 3A and 3B illustrate aspects of a pipetting system in accordance with the present invention, wherein sample transport from an output tray to an input tray may be facilitated with controllably illuminated tray substrates.

FIG. 4 illustrates aspects of a pipetting system in accordance with the present invention, wherein a controllably illuminated tray substrate is shown along with a tray in side view.

FIG. 5A illustrates aspects of a pipetting system in accordance with the present invention, wherein sample transport from an output tray to an input tray may be facilitated with controllably illuminated tray substrates, and wherein aspects of the memory, control, and lighting features may be physically coupled to one of the tray substrates.

FIG. 5B illustrates aspects of a pipetting system in accordance with the present invention, wherein sample transport from an output tray to an input tray may be facilitated with controllably illuminated tray substrates, and wherein aspects of the memory, control, and lighting features may be physically coupled to one of the tray substrates, while other aspects may be connected to each other via wireless communication.

FIG. 6 illustrates a process for facilitating controlled pipetting in accordance with the present invention.

FIG. 7 illustrates a process for facilitating controlled pipetting in accordance with the present invention, wherein a display, such as a computer monitor, may be utilized to graphically represent aspects of the pipetting process.

FIG. 8 illustrates a process for facilitating controlled pipetting in accordance with the present invention, wherein a display, such as a computer monitor, may be utilized to graphically represent aspects of the pipetting process, and wherein a sensor may be utilized to provide signals regarding various steps of the pipetting process.

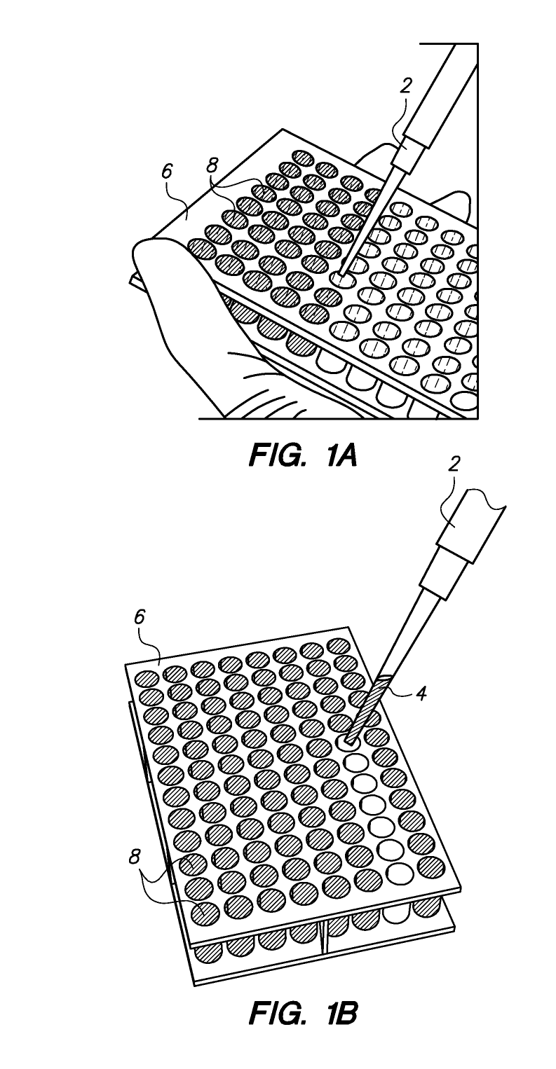

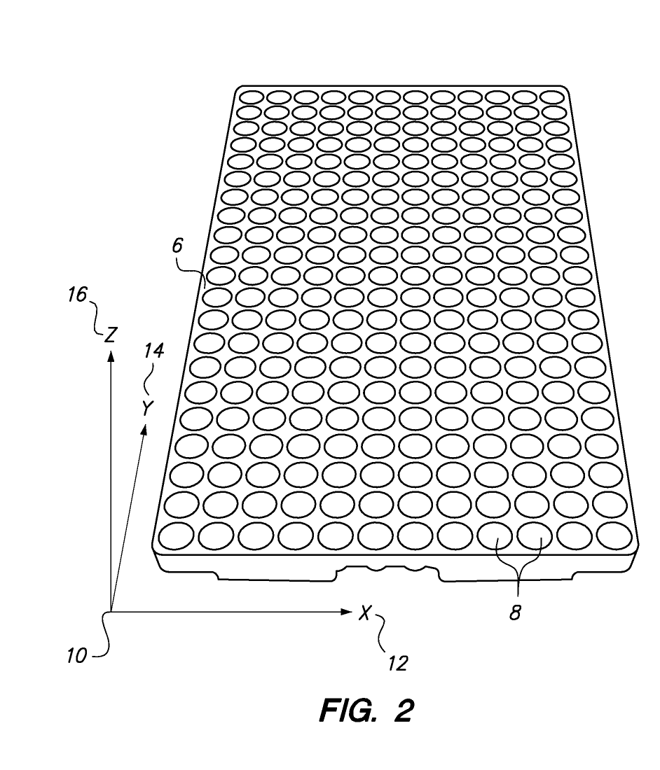

### SUMMARY

One embodiment is directed to a system for assisting with pipette-based substance transport between two or more trays, comprising: a first tray comprising a first matrix of wells; a first tray substrate removably coupled to the first tray, the first tray substrate comprising a first matrix of lights, each of which is aligned with and configured to be able to discretely illuminate a well of the first matrix of wells; a second tray comprising a second matrix of wells; a second tray substrate removably coupled to the second tray, the second tray substrate comprising a second matrix of lights, each of which is aligned with and configured to be able to discretely illuminate a well of the second matrix of wells; and a controller operatively coupled to each of the first matrix of lights and the second matrix of lights and configured to selectively and discretely illuminate one or more of the first matrix of wells and second matrix of wells subject to a predetermined instruction set contained on a memory device operatively coupled to the controller; wherein the predetermined instruction set is configured to direct a pipette operator, by discretely illuminating groupings of wells in the first and second matrices of wells, to sample substances from various wells of the first matrix of wells and dispose of them in various wells of the second matrix of wells. The first matrix of lights may comprise discrete light sources, and least one of the discrete light sources may be an LED light source. The second matrix of lights may comprise discrete light sources, and least one of the discrete light sources is an LED light source. An LED light source may be controlled to illuminate with two or more colors and/or two or more brightnesses. The system may further comprise an operator input device configured to send a signal to the controller indicating that an additional step following the predetermined instruction set has been completed. The controller may be configured to change a combination of lighting of the first matrix of lights and the second matrix of lights in accordance with completion of the additional step, subject to the predetermined instruction set. The system may comprise one or more lighting power systems operatively coupled to either of the first and second matrices of lights, and to the controller, the one or more lighting power systems configured to provide controlled power to the lights subject to the controller and predetermined instruction set. The predetermined instruction set may comprise a set of comma separated values stored on the memory device. The predetermined instruction set may comprise one or more lines of computer code stored on the memory device. The input device may be selected from the group consisting of: a footpedal, a mouse, a button, a touchscreen, a microphone, and a button on a pipetting device. The memory device may be coupled to one of the first or second trays. The memory device may be coupled to one of the first or second trays. The input device may be coupled to the controller by a wired connection. The input device may be coupled to the controller by a wireless connection. A completed well may be designated by illumination into such well by the pertinent tray substrate.

Another embodiment is directed to a method for assisting with pipette-based substance transport between two or more trays, comprising illuminating one or more discrete wells of a first tray comprising a first matrix of wells, and one or more discrete wells of a second tray comprising a second matrix of wells, using a first controllably illuminated tray substrate removably coupled to the first tray, and a second controllably illuminated tray substrate removably coupled to the second tray, each of the controllably illuminated tray substrates comprising a matrix of lights aligned with and configured to be able to discretely illuminate a well of the adjacent matrix of wells; wherein a controller operatively coupled to each of the matrices of lights controls the illumination of the lights subject to a predetermined instruction set contained on a memory device operatively coupled to the controller. The first matrix of lights may comprise discrete light sources, and least one of the discrete light sources may be an LED light source. The second matrix of lights may comprise discrete light sources, and least one of the discrete light sources may be an LED light source. An LED light source may be controlled to illuminate with two or more colors and/or two or more brightnesses. The method may further comprise receiving an operator input signal from an operator input device indicating that an additional step following the predetermined instruction set has been completed. The method may further comprise changing a combination of lighting of the first matrix of lights and the second matrix of lights in accordance with completion of the additional step, subject to a predetermined instruction set. The method may further comprise coupling one or more lighting power systems to either of the matrices of lights, and to the controller, the one or more lighting power systems being configured to provide controlled power to the lights subject to the controller and predetermined instruction set. The predetermined instruction set may comprise a set of comma separated values stored on the memory device. The predetermined instruction set may comprise one or more lines of computer code stored on the memory device.

The input device may be selected from the group consisting of: a footpedal, a mouse, a button, a touchscreen, a microphone, and a button on a pipetting device. The method may further comprise coupling a memory device to one of the two or more trays. The method may further comprise coupling the controller to one of the two or more trays. The method may further comprise coupling the input device to the controller with a wired connection. The method may further comprise coupling the input device to the controller with a wireless connection. The method may further comprise illuminating the one or more discrete wells in response to a signal designating such wells as completed.

### DETAILED DESCRIPTION

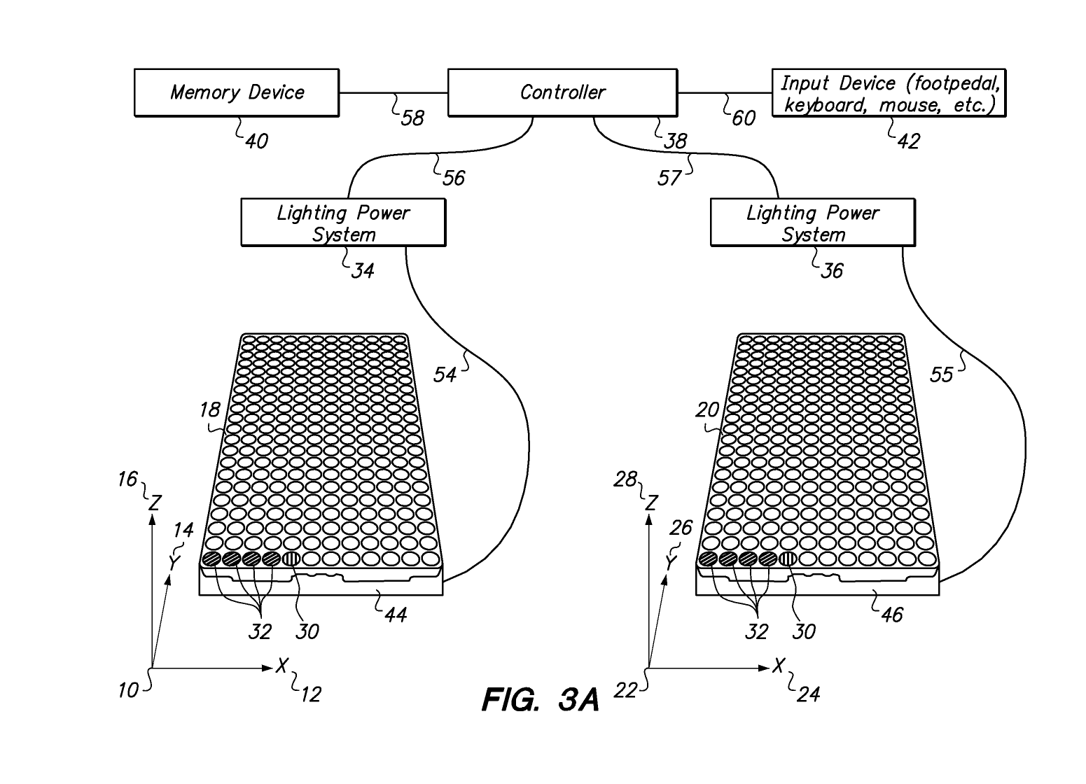

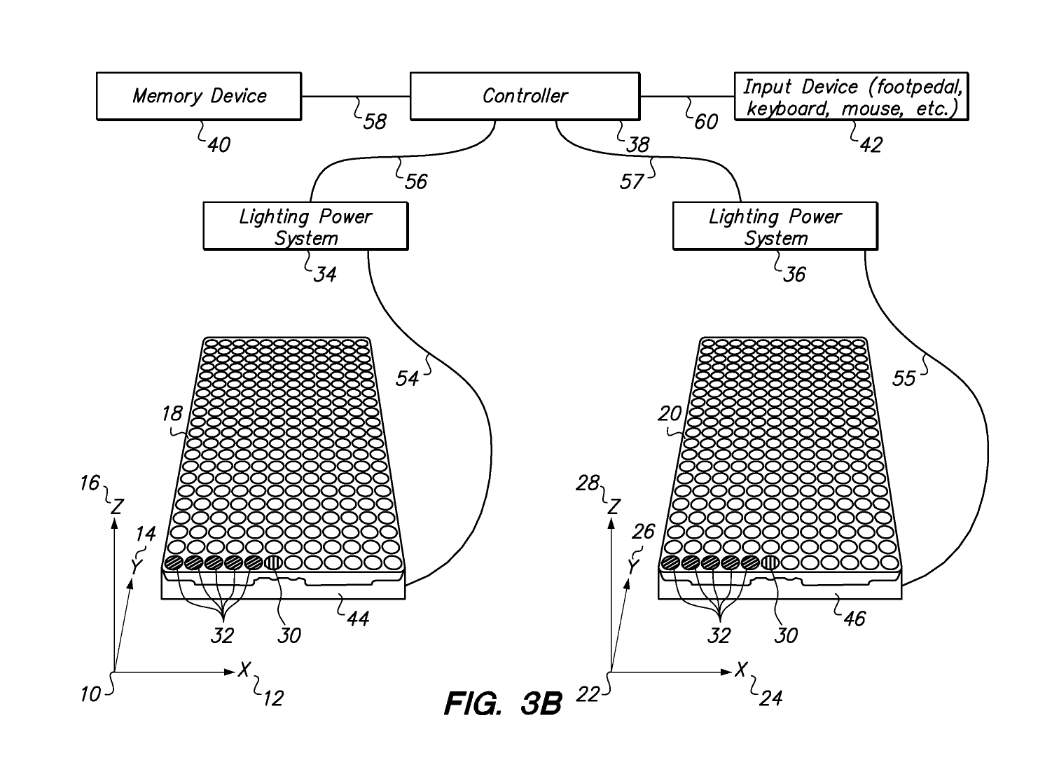

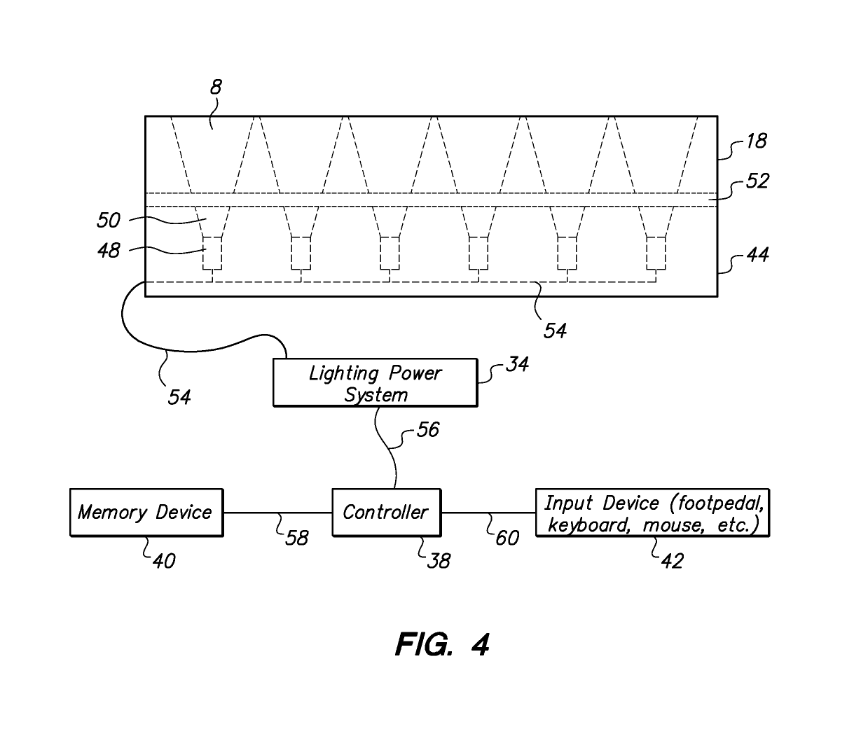

Referring to FIG. 3A, a configuration is depicted wherein a source or output tray (18) is placed adjacent a destination or input tray (20). Removably coupled to each of these trays (18, 20), is a controllably illuminated substrate (44, 46) configured to be able to discretely illuminate particular wells of each of the trays (18, 20). Referring ahead to FIG. 4, a side view of a tray (18) removably coupled to an illumination substrate (44) is depicted to illustrate that the illumination substrate (44) comprises a series of light sources (48), such as light emitting diodes (“LED”), conventional resistive light bulbs, or other lighting elements that may be controllably switched on and off, and preferably that may be switched on in more than one color, and more than one brightness. For example, in one embodiment, each lighting element (48) comprises one or more LEDs capable of not only controllably switching off, but also switching on with a variety of brightnesses distinguishable to an operator, and also a number of colors distinguishable to an operator (in one embodiment through a small bundle of different colors of LED elements packaged together as a single lighting element 48). In one embodiment, an LED element bundle may comprise LED elements capable of producing light with three or more colors simultaneously at varying brightnesses (e.g., one red LED element, one green LED element, and one blue LED element, each of which having independent brightness control)—thus enabling such bundle to produce any hue and any brightness.

In the depicted embodiment, each lighting element (48) is configured to be a source of light that is broadcasted or emitted upward through a light well (50) toward an associated discrete tray well (8). In other words, in the preferred embodiment, lighting elements (48) and wells (8) are matched on a one-to-one basis; this prevents potential errors that may arise (for, example, due to unclarity in an operator's perception as to which well is being lit) if one element is associated with two or more wells—or if a larger lighting element, such as an LCD flat panel, is used as a lighting substrate. Referring again to FIG. 4, as light passes through the light well (50) toward the tray well (8), it is transmitted across an isolation layer (52) which may be configured to provide thermodynamically insulative properties. For example, in an embodiment wherein one or more resistive lighting elements (48) are utilized, and wherein samples with in the tray wells (8) may be sensitive to temperature increases which may be associated to small heat sources such as resistive lighting elements, the isolation layer (52) may be selected, and geometrically sized (i.e., in thickness), to not only transmit light, but also to prevent the transmission of heat. Also shown in FIG. 4 is a lighting power system (34) coupled to the lighting elements by a wire lead (54). The lighting power system (54) may be coupled via another wire lead (56) to a controller (38), such as a microprocessor (i.e., which may be housed in a computing system such as a laptop computer) or microcontroller, which is coupled via another conductive connection (58) to a memory device (40), such as a hard drive, flash memory device, or the like. The controller (38) also is depicted coupled by another wire lead (60) to an operator command input device (42), such as a footpedal, keyboard, mouse device, touchscreen, button on a pipetting device, etc., which may be utilized to advance a program operating on the controller (38) to a next step of execution of a program or predetermined instruction set.

Referring back to FIG. 3A, a configuration is depicted wherein an output tray (18) is removably coupled to one illumination substrate (44) akin to that depicted in side view in FIG. 4. An input tray (20) is similarly removably coupled to another illumination substrate (46), and the input tray (20) is illustrated with its own coordinate system (22) (X 24, Y 26, and Z 28). Each of the illumination substrates are coupled via a conductive lead (54, 55) to a lighting power system (34, 36), which is coupled via a conductive lead (56, 57) to the controller (38). In another embodiment, a single lighting power system may be coupled to both, of power subsystems may be embedded with the illumination substrates. To assist an operator in removing a sampled substance portion from selected wells of the output tray (18) and getting them into the correct predetermined wells of the input tray (20), an indication of the two active wells (one as the source, one as the receiving well) may be displayed for the operator, such as a relatively bright illumination level or color (30). A different visual indication (32), such as a less bright color or level of illumination, may be utilized to indicate for the operator that certain wells already been utilized. Referring to FIG. 3B, with one input from the input device (42), the controller (38) may be configured to advance the lighting configuration of the substrates (44, 46) to indicate that the next predetermined pair of wells are now active (30), along with an indication regarding wells that have been completed (32). A predetermined plan, comprising, for example, computer code, lookup table information, arrays of coordinates, and/or lists of comma separated values, may be stored on the memory device (40) and made accessible to the controller for advancement in a step by step fashion, subject to inputs from the operator that each additional step has been completed.

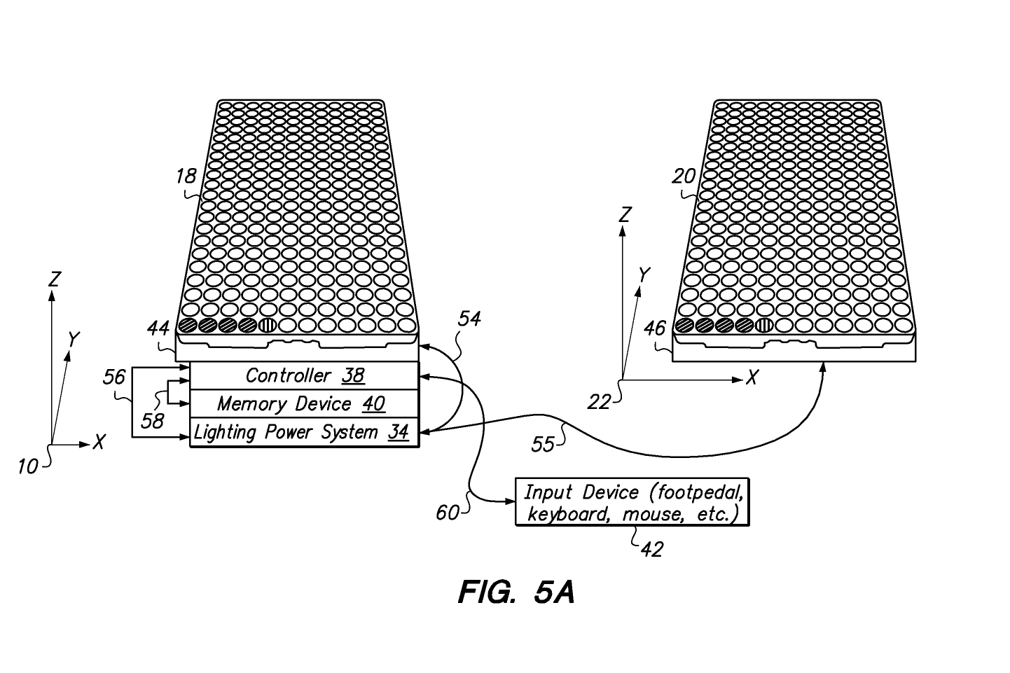

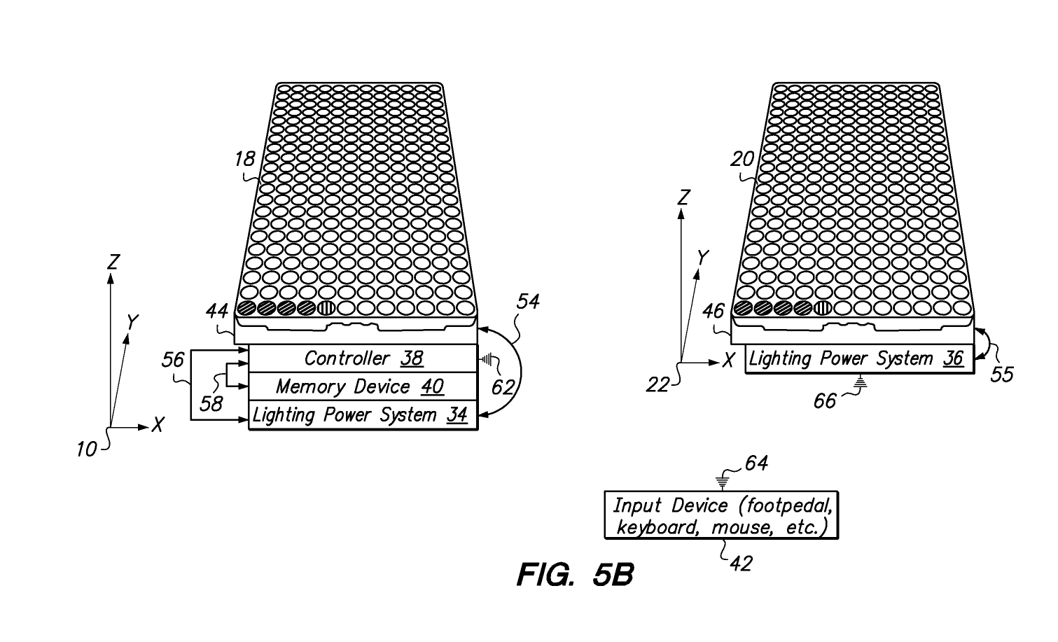

Referring to FIG. 5A, in another embodiment, the controller (38), memory device (40), and at least one lighting power system (34) may be coupled to one of the illumination substrates (44) with leads (55, 60) to other elements of the functional system. Referring to FIG. 5B, in another embodiment, one or more direct conductive leads may be replaced with wireless communication links between various elements. For example, in the illustrated embodiment, the controller (38), memory device (40), and lighting power system (34) remain coupled to one of the illumination substrates (44) while connections between the other lighting power system (36), the input device (64), and the controller may be conducted with wireless transmitter/receiver devices (62, 64, 66) coupled thereto.

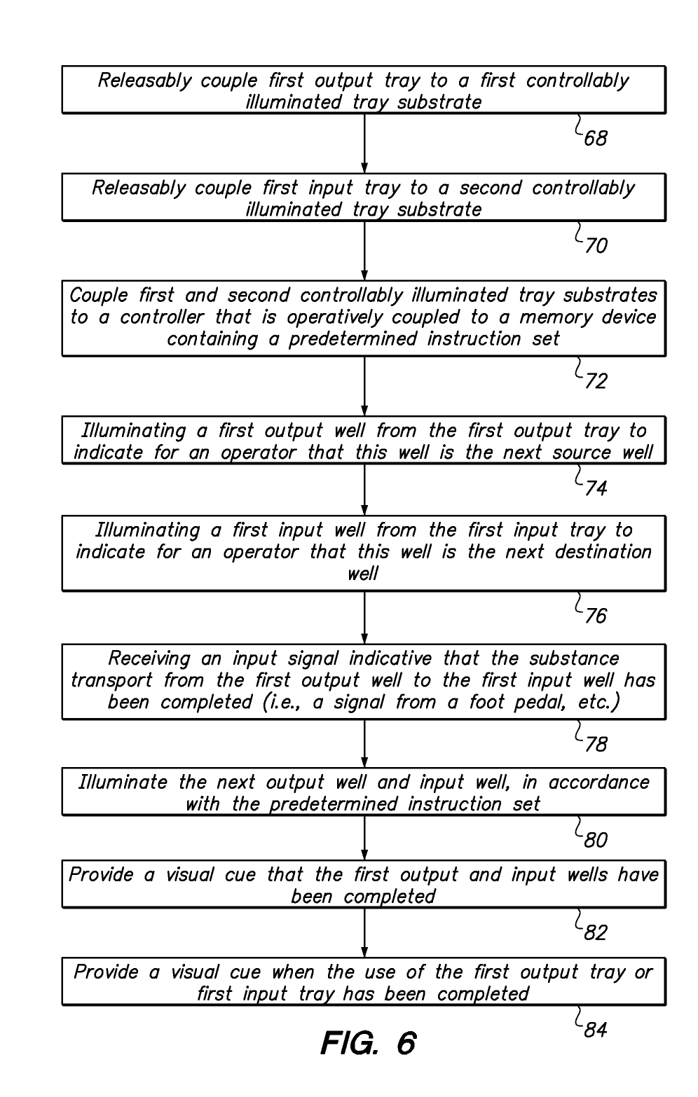

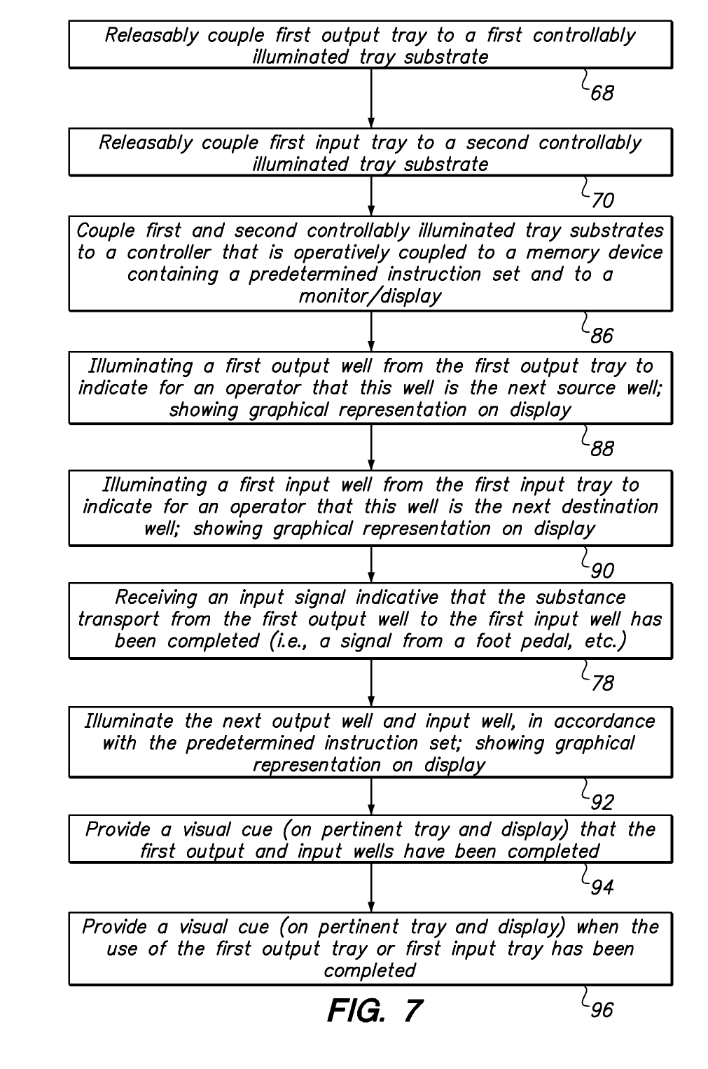

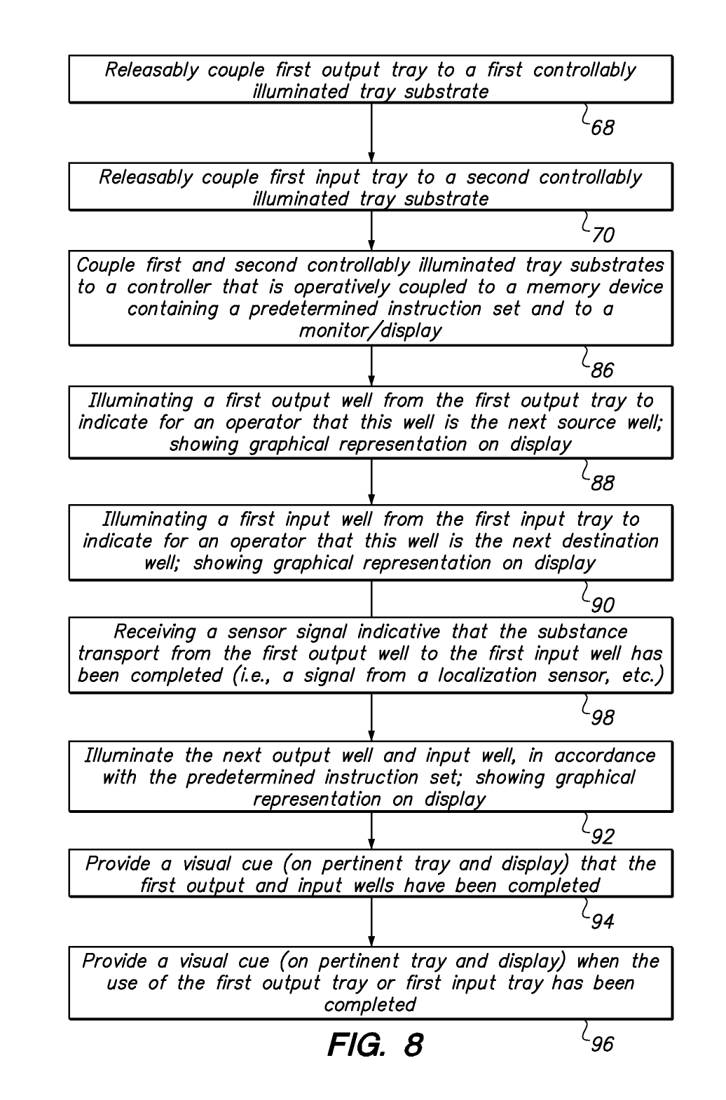

Referring to FIG. 6, a process in accordance with the present invention is illustrated, wherein an output tray is releasably coupled to an illumination substrate (68) and an input tray is releasably coupled to an illumination substrate (70), such substrates are coupled to a controller coupled to a memory device containing a predetermined instruction set (72), and a pipetting operation begun. In accordance with the predetermined instruction set, a first output well may be discretely illuminated (74) along with a first input well (76). After the two active wells have been serviced by the operator directing the pipette, the operator may use the input device to produce a signal that may be received by the controller as a sign that the particular cycle has been completed (78), and the next two active wells may be illuminated (80) in accordance with the instruction set. In one embodiment, a visual cue may be provided to signal to the operator that the two previously active wells have been completed (82), such as a quick blinking of those cells followed by illumination of the next active pair. Further, another visual cue, such as illumination or blinking of the entire illumination matrix (i.e., every LED on the substrate), may be utilized to signal to the operator that the program or instruction set for that particular pairing of trays has been exhausted (i.e., it is time to switch to another input tray, output tray, or pair of trays). In another embodiment (not shown), similar configurations may be utilized to coordinate the pipetting of more than two trays. For example, in one embodiment, substances may be moved from one output tray to two destination trays, and illumination substrates may be so coordinated. In another embodiment, as illustrated by FIG. 7, a display or monitor (such as a computer display) may comprise a graphical user interface configured to mimic for a viewer the activity going on with the pertinent trays and illumination substrates.

Referring to FIG. 7, after the trays are coupled to the pertinent illumination substrates (68, 70), the controller may be operatively coupled not only to the substrates to output indications to the operator - but also may be coupled to a display or monitor system (86). The illumination of wells using the lighting substrate may also be shown in the display (88, 90, 92), as well as illumination configurations for visual cues (94, 96).

Referring to FIG. 8, a configuration similar to that of FIG. 7 is illustrated, with exception that a sensor configuration is present to sense when each subsequent step of pipetting has been completed (i.e., in this embodiment, the manual input device advancement by the operator is not needed—because it is automated using the sensor). For example, in one embodiment, a localization sensor may track in three dimensions the location of a distal tip of a pipette—and another sensing element may be configured to confirm that a substance has been captured and/or removed. In another embodiment, sensors may be configured to establish that a distal tip of a pipette has crossed the circular threshold of a given well (for example, by having a simple loop circuit around each well, and a ferromagnetic metal piece in the pipette tip that creates a small but detectable current as the metal piece is passed through each circuit loop). Such circuit loops may be embedded into one of more trays, or may comprise a sterile printed circuit overlay layer that is positioned on top of a given tray.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject interventions may be provided in packaged combination for use in executing such interventions. These supply “kits” further may include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more coatings (e.g., hydrophilic polymers such as polyvinylpyrrolidone-based compositions, fluoropolymers such as tetrafluoroethylene, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

1. A system for assisting with pipette-based substance transport between two or more trays, comprising:
a. a first tray comprising a first matrix of wells;
b. a first tray substrate removably coupled to the first tray, the first tray substrate comprising a first matrix of lights, each of which is aligned with and configured to be able to discretely illuminate a well of the first matrix of wells;
c. a second tray comprising a second matrix of wells;
d. a second tray substrate removably coupled to the second tray, the second tray substrate comprising a second matrix of lights, each of which is aligned with and configured to be able to discretely illuminate a well of the second matrix of wells; and
e. a controller operatively coupled to each of the first matrix of lights and the second matrix of lights and configured to selectively and discretely illuminate one or more of the first matrix of wells and second matrix of wells subject to a predetermined instruction set contained on a memory device operatively coupled to the controller;
wherein the predetermined instruction set is configured to direct a pipette operator, by discretely illuminating groupings of wells in the first and second matrices of wells, to sample substances from various wells of the first matrix of wells and dispose of them in various wells of the second matrix of wells.

2. The system of claim 1, wherein the first matrix of lights comprises discrete light sources.

3. The system of claim 2, wherein at least one of the discrete light sources is an LED light source.

4. The system of claim 1, wherein the second matrix of lights comprises discrete light sources.

5. The system of claim 4, wherein at least one of the discrete light sources is an LED light source.

6. The system of claim 3, wherein the LED light source may be controlled to illuminate with two or more colors.

7. The system of claim 5, wherein the LED light source may be controlled to illuminate with two or more colors.

8. The system of claim 3, wherein the LED light source may be controlled to illuminate with two or more brightnesses.

9. The system of claim 5, wherein the LED light source may be controlled to illuminate with two or more brightnesses.

10. The system of claim 1, further comprising an operator input device configured to send a signal to the controller indicating that an additional step following the predetermined instruction set has been completed.

11. The system of claim 10, wherein the controller is configured to change a combination of lighting of the first matrix of lights and the second matrix of lights in accordance with completion of the additional step, subject to the predetermined instruction set.

12. The system of claim 1, further comprising one or more lighting power systems operatively coupled to either of the first and second matrices of lights, and to the controller, the one or more lighting power systems configured to provide controlled power to the lights subject to the controller and predetermined instruction set.

13. The system of claim 1, wherein the predetermined instruction set comprises a set of comma separated values stored on the memory device.

14. The system of claim 1, wherein the predetermined instruction set comprises one or more lines of computer code stored on the memory device.

15. The system of claim 1, wherein the input device is selected from the group consisting of: a footpedal, a mouse, a button, a touchscreen, a microphone, and a button on a pipetting device.

16. The system of claim 1, wherein the memory device is coupled to one of the first or second trays.

17. The system of claim 1, wherein the controller is coupled to one of the first or second trays.

18. The system of claim 1, wherein the input device is coupled to the controller by a wired connection.

19. The system of claim 1, wherein the input device is coupled to the controller by a wireless connection.

20. The system of claim 1, wherein a completed well is designated by illumination into such well by the pertinent tray substrate.

21. A method for assisting with pipette-based substance transport between two or more trays, comprising:
illuminating one or more discrete wells of a first tray comprising a first matrix of wells, and one or more discrete wells of a second tray comprising a second matrix of wells, using a first controllably illuminated tray substrate removably coupled to the first tray, and a second controllably illuminated tray substrate removably coupled to the second tray, each of the controllably illuminated tray substrates comprising a matrix of lights aligned with and configured to be able to discretely illuminate a well of the adjacent matrix of wells;
wherein a controller operatively coupled to each of the matrices of lights controls the illumination of the lights subject to a predetermined instruction set contained on a memory device operatively coupled to the controller.

22. The method of claim 21, wherein the first matrix of lights comprises discrete light sources.

23. The method of claim 22, wherein at least one of the discrete light sources is an LED light source.

24. The method of claim 21, wherein the second matrix of lights comprises discrete light sources.

25. The method of claim 24, wherein at least one of the discrete light sources is an LED light source.

26. The method of claim 23, wherein the LED light source may be controlled to illuminate with two or more colors.

27. The method of claim 25, wherein the LED light source may be controlled to illuminate with two or more colors.

28. The method of claim 23, wherein the LED light source may be controlled to illuminate with two or more brightnesses.

29. The method of claim 25, wherein the LED light source may be controlled to illuminate with two or more brightnesses.

30. The method of claim 21, further comprising receiving an operator input signal from an operator input device indicating that an additional step following the predetermined instruction set has been completed.

31. The method of claim 30, further comprising changing a combination of lighting of the first matrix of lights and the second matrix of lights in accordance with completion of the additional step, subject to a predetermined instruction set.

32. The method of claim 21, further comprising coupling one or more lighting power systems to either of the matrices of lights, and to the controller, the one or more lighting power systems being configured to provide controlled power to the lights subject to the controller and predetermined instruction set.

33. The method of claim 21, wherein the predetermined instruction set comprises a set of comma separated values stored on the memory device.

34. The method of claim 21, wherein the predetermined instruction set comprises one or more lines of computer code stored on the memory device.

35. The method of claim 30, wherein the input device is selected from the group consisting of: a footpedal, a mouse, a button, a touchscreen, a microphone, and a button on a pipetting device.

36. The method of claim 21, further comprising coupling a memory device to one of the two or more trays.

37. The method of claim 21, further comprising coupling the controller to one of the two or more trays.

38. The method of claim 30, further comprising coupling the input device to the controller with a wired connection.

39. The method of claim 30, further comprising coupling the input device to the controller with a wireless connection.

40. The method of claim 21, further comprising illuminating the one or more discrete wells in response to a signal designating such wells as completed.
